(19) United States
(12) Patent Application Publication
(10) Pub. No.: US 2025/0230393 A1
(43) Pub. Date: Jul. 17, 2025

(54) OBSERVATION CHAMBER FOR A WELL ARRAY CHIP

(71) Applicant: Teracyte Ltd., Yokneam (IL)

(72) Inventors: Yoav NISSAN-COHEN, Mishmar David (IL); Aviram COHEN, Galia (IL); Shay KAPLAN, Givat Elah (IL)

(73) Assignee: Teracyte Ltd., Yokneam (IL)

(21) Appl. No.: 19/014,734

(22) Filed: Jan. 9, 2025

Related U.S. Application Data
(60) Provisional application No. 63/619,898, filed on Jan. 11, 2024.

(51) Int. Cl. C12M 1/00; C12M 1/12; C12M 3/00

(52) U.S. Cl. CPC C12M 23/22; C12M 23/04; C12M 23/34; C12M 23/48; C12M 29/16; C12M 29/20

(57) ABSTRACT

An observation chamber is presented. The observation chamber includes a bottom part configured to accept a well array chip; one or more sidewalls extending from the bottom part; a top part comprising a viewing glass; wherein the bottom part, the one or more sidewalls and the top part enclose therein a media chamber; and wherein the media chamber is configured to receive a biological media such that the viewing glass is submerged therein. Consequently, continuous monitoring of biological cells trapped in well array chip is achieved, maintaining a high image quality, while biological media covers the well array chip for extended periods.

### CROSS-REFERENCE TO RELATED APPLICATIONS

This application claims the benefit of U.S. Provisional Application No. 63/619,898 filed Jan. 11, 2024, the contents of which are hereby incorporated by reference for all that it contains.

### TECHNICAL FIELD

The present disclosure relates generally to well array chips placed within biological media, and more particularly to a chamber wherein a well array chip is placed therein for application of a biological media for the purpose of observation of cells trapped in the wells of the well array chip.

### BACKGROUND

Use of array chips that contain a plurality of wells adapted to capture therein biological cells has been known for a while. The well array chip, or carrier chip, contains an array of hundreds, thousands, or tens of thousands of wells, arranged in a two-dimensional array, the wells protruding into the carrier chip that provides their physical integrity. The well array chips are used in research, development, and testing of biological cells in multiple ways.

One desirable way is the continuous inspection of live cells trapped in well array chips and immersed in media, which has many uses. There are many advantages to performing these measurements using inverted microscopes. Since cell viability must be retained over time, the well array chips should be covered by biological media (also referred to herein as media) which is replenished continuously. That means that there is a need for a media to be present over the well array chip.

However, there is a major problem in this configuration as it is very difficult to ensure uniformity of the media layer thickness. Since focal distance is affected by the thickness of the media layer, nonuniformity makes it difficult to ensure image quality. The challenge of current systems is how to ensure high image quality while keeping cells viability over time. Image quality is paramount if reliable and consistent results are to be achieved.

It would be therefore advantageous to provide a solution that overcomes the deficiencies noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “certain embodiments” may be used herein to refer to a single embodiment or multiple embodiments of the disclosure.

Some example embodiments disclosed herein include an observation chamber comprising: a bottom part configured to accept a well array chip; one or more sidewalls extending from the bottom part; a top part comprising a viewing glass; wherein the bottom part, the one or more sidewalls and the top part enclose therein a media chamber; and wherein the media chamber is configured to receive a biological media such that the viewing glass is submerged therein.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a three-dimensional illustration of a bottom part of a perfusion observation chamber for a well array chip according to an embodiment.

FIG. 2 is a three-dimensional illustration of a top part of a perfusion observation chamber for a well array chip according to an embodiment.

FIG. 3 is a cross-section of the top part of a perfusion observation chamber for a well array chip according to an embodiment.

FIG. 4 is a cross-section of the bottom part of a perfusion observation chamber of a well array chip according to an embodiment.

FIG. 5 is a cross-section of the perfusion observation chamber when the top part is placed on top of the bottom part according to an embodiment.

FIGS. 6A-6F depict various illustrations of a perfusion observation chamber and parts thereof according to another embodiment.

FIG. 7 is a schematic block diagram of a system employing a perfusion observation chamber according to another embodiment.

FIG. 8 is a three-dimensional illustration of a perfusion observation chamber for a well array chip according to another embodiment.

FIG. 9 is a flowchart for an operation of the system employing a perfusion observation chamber according to another embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

An observation chamber comprises a bottom part configured to accept a well array chip; one or more sidewalls extending from the bottom part; a top part comprising a viewing glass; wherein the bottom part, the one or more sidewalls and the top part enclose therein a media chamber; and wherein the media chamber is configured to receive a biological media such that the viewing glass is submerged therein. Consequently, continuous monitoring of biological cells trapped in the well array chip is achieved, maintaining a high image quality, while biological media covers the well array chip for extended periods.

Reference is now made to FIG. 1 which is an example three-dimensional illustration of a bottom part 100 of a perfusion observation chamber of a well array chip according to an embodiment. The bottom part 100 includes a body 110 which has a floor 115 and four side walls 111, 112, 113, and 114 extending at an angle, for example, but not by way of limitation, a ninety-degree angle, therefrom. Side walls 112 further includes an inlet 120 adapted to accept flow of biological media, referred to herein also simply as media, into the hollow created by the four side walls 111 through 114 and the floor 115. It should be understood that, while a rectangular shape is formed by the four side walls 111 through 114, other polygons may be used without departing from the scope of the disclosed embodiments, and further a circular sidewall is also possible. The top part 200, further explained herein, shall match in shape so as to fit properly into the bottom part 100.

In an embodiment, the inlet 120 may protrude the side wall 112 at an angle. In an embodiment, the inlet 120 may be positioned and protrude to the top part 200. It should be further understood that one or more inlets 120 may be used, for example, to allow for the flow of different types of media which may not, for example, be mixed prior to entering into the perfusion observation chamber. An outlet 130 for the media which is adapted to ensure that at least a desired minimum level of media covers the floor 115, and, as further explained herein, provides an escape route to allow air bubbles to escape through. The floor 115 is further adapted to accept a well array chip 140, the well array chip including a plurality of wells adapted to trap therein cells. In an embodiment a plurality of receptors 150, for example, receptors 150-1, 150-2, 150-3, and 150-4 are provided, designed to accept a matching peg to ensure proper connection and/or lock between the bottom part 100 and the top part 200, further discussed herein in more detail. In an embodiment, the well array chip 140 is secured to the floor 115, for example, but not by way of limitation, by one or more hinges (not shown).

The bottom part 100 and the top part 200 are designed to fit together to create a perfusion observation chamber. It should be noted that though a cuboid-like chamber is shown, other forms may be used, for example, and without limitation, a cylinder, cube, cuboid, or designs having other polygonal floor shapes, such as a hexagon, without departing from the scope of the disclosed embodiments. In an embodiment, the internal portions of the side walls 111 through 114 and the floor 115, together forming the perfusion chamber, are coated by a hydrophobic layer that is adapted to repel the media flowing in and out of the perfusion chamber.

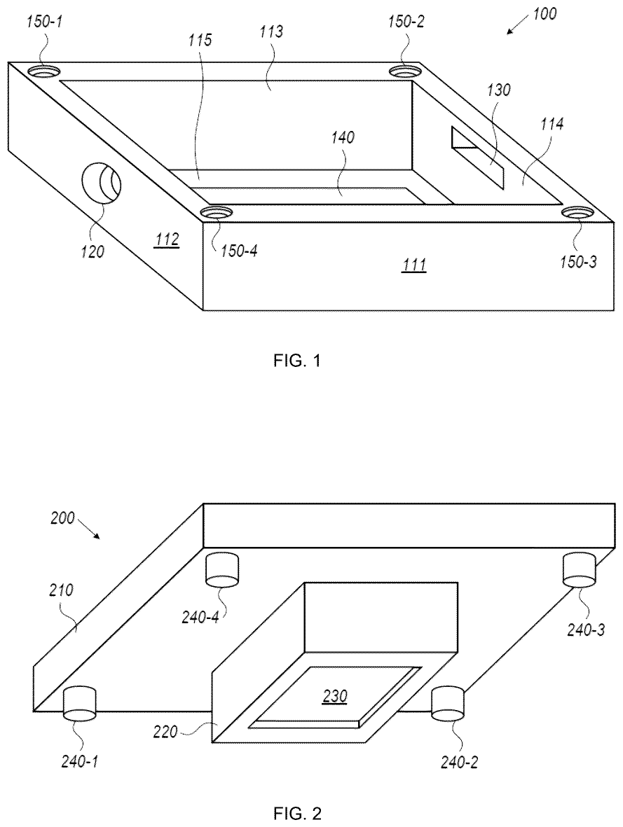

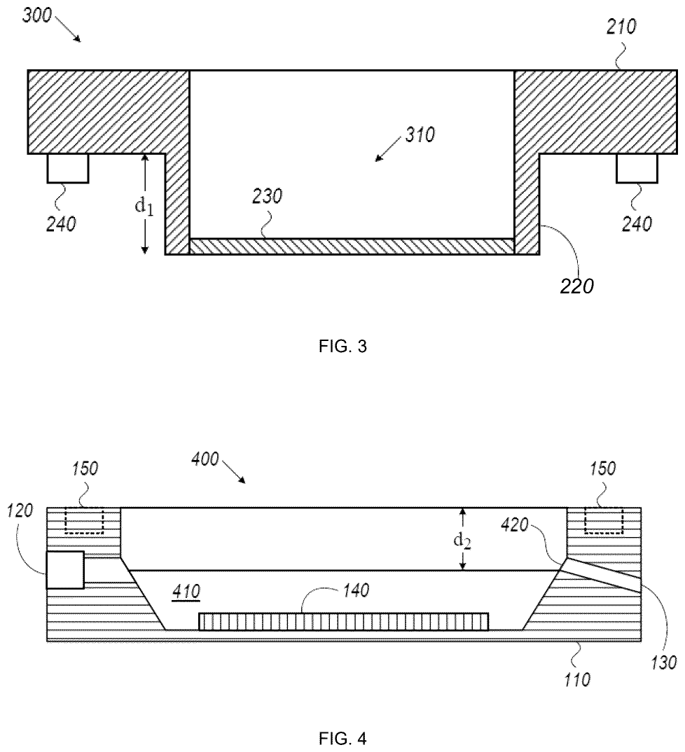

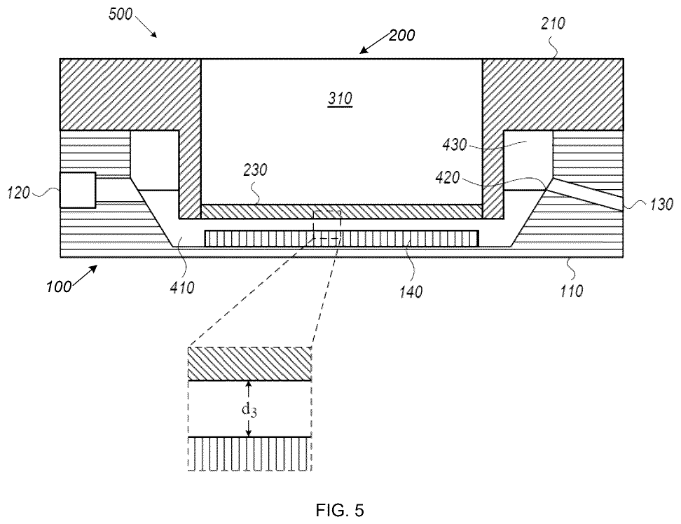

FIG. 2 is an example three-dimensional illustration of a top part 200 of a perfusion observation chamber of a well array chip according to an embodiment. The top part 200 includes roof 210 the center of which (not shown in this figure but see FIGS. 3 and 5) has an opening (see 310 in FIG. 3) that protrudes through the roof 210. To the bottom of the roof 210, and in alignment with the protruding opening, there is formed an observation chamber 220, the bottom of which is equipped with a viewing glass 230. It should be understood that the viewing glass 230 may include a variety of optically transparent materials, including but not limited to glass. In an embodiment, the viewing glass 230 may be coated, on one or both sides, with one or more layers of coating. In an embodiment, this coating layer may be a hydrophobic material. In another embodiment, the coating layer may be an optical filter, where such filter may be a low-pass filter, high-pass filter, band pass filter, and notch pass (band reject) filter. In an embodiment a wide band pass anti-reflective coating, that covers both visible and near infra-red (NIR), having a wavelength in the range of 350-1000 nm, If we use wide band ARC that covers both Visible and NIR (350-1000 nm), may be used.

Any one of a plurality of fastening mechanisms may be used to fasten the top part 200 to the bottom part 100. In an embodiment a plurality of pegs 240, for example, pegs 240-1, 240-2, 240-3, and 240-4, are attached to the bottom of the roof 210 so that when the top part 200 is placed on the bottom part 100 the pegs 240 align within the receptors 150. In an embodiment, the pegs 240 are of magnetic, such that when the receptors 150 are of ferromagnetic material an attraction will cause the top part 200 to lock, preferably tightly, with the bottom part 100, and the reverse is also possible. While pegs 240 are shown, it should be understood that other fastening techniques of the top part 200 to the bottom part 100 are possible. For example, screws and bolts (not shown) may be used instead of the pegs 240 which may either be screwed in place or snapped into place as the case may be. In an embodiment, a seal (not shown), for example, made of rubber, is placed around the permitter of at least one of the top part 200 and the bottom part 100 where contact is made to ensure quality sealing so that media cannot leak at their contact points.

One of ordinary skill in the art would readily appreciate that either of the top part 200 and the bottom part 100 may each include a plurality of subparts without departing from the scope of the disclosed embodiments. Moreover, certain subparts of the top part 200 may be included as part of the bottom part 100 and vice versa without departing from the scope of the disclosed embodiments. The top part 200 and the bottom part 100 may be manufactured in any kind of manufacturing process, including but not limited to, molding-based process and 3D printing, without departing from the scope of the disclosed embodiments.

FIG. 3 is an example cross-section 300 of the top part 200 of a perfusion observation chamber of a well array chip according to an embodiment. The roof of the top part 210 is protruded by an opening to form a chamber 310 within the observation chamber 220. A viewing glass 230 is mounted at the bottom of the chamber 310 of the top part 200. The observation chamber 220 extends from the bottom of the roof 210 to a distance marked a d₁, the importance of which shall be discussed with respect to FIGS. 4 and 5 herein.

FIG. 4 is an example cross-section 400 of the bottom part 100 of a perfusion observation chamber of a well array chip 140 according to an embodiment. An inlet 120 provides for an input of biological media that is to cover the well array chip 140. As it needs to be constantly renewed, an outlet 130 is used as an exit channel, wherein the exit 420 is adapted to ensure that the level of the media reaches a distance of at least d₂ from the top surface of the bottom part 100. The distance d₂ is smaller than the distance d₁, i.e., d₁>d₂, so that, as further shown in FIG. 5 herein, the bottom of the viewing glass 230 is below the surface of the media, i.e., the viewing glass is submerged with respect to the media within the media chamber 410. The design of exit 420 of the outlet 130 provides an escape route for any air bubbles that may appear under the viewing glass 230, once submerged, thereby ensuring high-quality imaging from the observation chamber 310.

FIG. 5 is an example cross-section 500 of the perfusion observation chamber when the top part 200 is placed on top of the bottom part 100 according to an embodiment. Once the top part 200 is placed on the bottom part 100 the media chamber 410 is formed, and the observation chamber 310 is placed over the well array chip 140 and viewable through the viewing glass 230. Media flowing from the inlet 120 through the media chamber 410 exits the media chamber through the outlet 130, where the edge of the outlet 130 provides for a level of media that is above the viewing glass level within the media chamber leaving a ds wide gap to allow for media to flow over the well array chip 140. Any bubbles that may develop under the surface of the viewing glass will flow with the media and exit through the outlet 130 which provides an escape path for such air bubbles. In an embodiment, the draining pipe is connected to a pump (not shown), for example, a vacuum pump, that allows to pump out media from the media chamber 410.

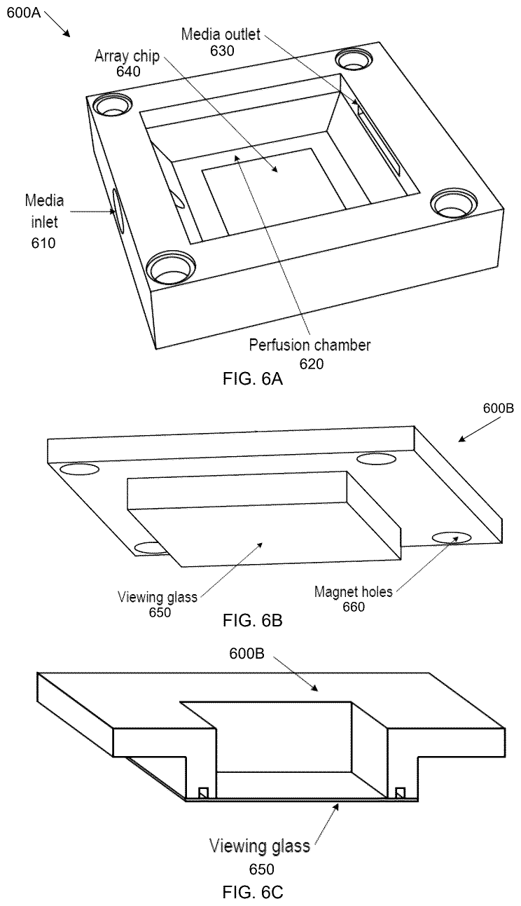

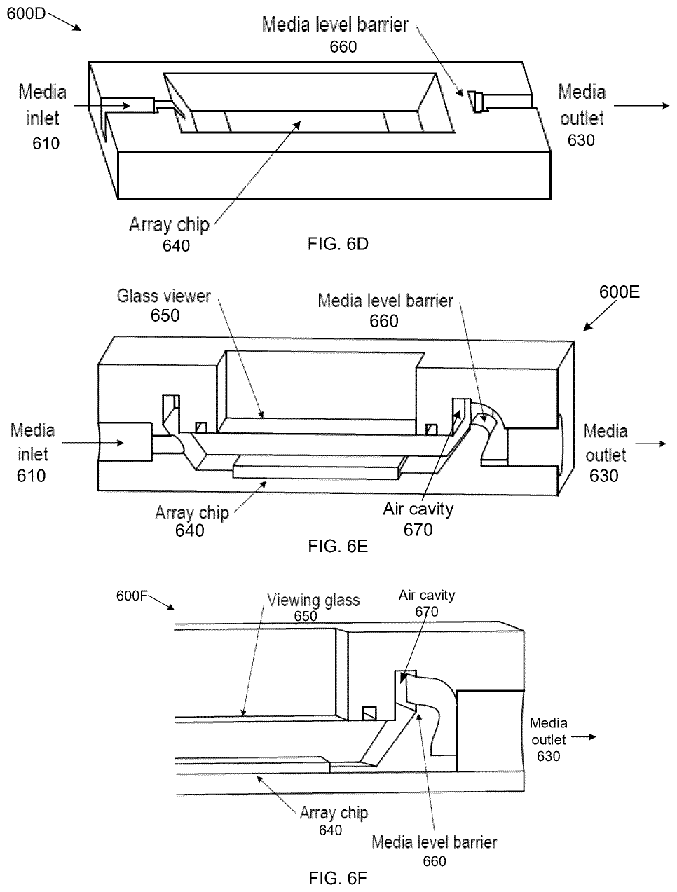

FIGS. 6A-6F depict various example illustrations of a perfusion observation chamber and parts thereof according to another embodiment. As noted, to keep cells alive over a long time, they are immersed in a biological media; thus, a well array chip 640 is placed in a kind of a bathtub, as presented in the three-dimensional illustration FIG. 6A of a bottom part 600A. Since the media is to be replenished continuously, an inlet 610 (marked as media inlet) for feeding fresh media to the perfusion chamber 620, and an outlet 630 (marked media outlet) are added for releasing used media to a draining pipe (not shown). It is crucial to control the depth (d₃) of the media layer above the well array chip 640 to ensure that cells are kept in focus to achieve the optimal image quality. In an embodiment, a marker on edge of outlet 630 may indicate the minimal level of media desired over the well array chip 640.

The depth d₃ of the media layer is accurately controlled by submerging a viewing glass 650 in the media above the well array chip 640, which ensures that the optical path throughout the well array chip 640 area is uniform. With this viewing glass 650, an optimal focus is obtained for the entire well array chip 640 area. A schematic three-dimensional design of a top part 600B with a viewing glass 650 is presented in FIG. 6B, an example cross-section of which is shown in FIG. 6C. The top part 600B and the bottom part 600A may be affixed to each other using magnets placed in magnet holes 660.

It should be appreciated that in current solutions when a chamber is filled with media, it is very probable that air bubbles are formed. This may become a major obstacle since these air bubbles can be formed under the viewing glass and obstruct the optical path, thus resulting in a deteriorated image quality.

In an embodiment, shown in FIGS. 6D and 6E air bubbles are eliminated by keeping an empty air cavity 670 above the media. Air bubbles move upward and are released from the media into the air cavity 670, providing an effective escape path for bubbles. Excess air is removed from the air cavity by keeping a small gap between the chamber body and cover. In this embodiment, air cavity 430 is formed inside the chamber body by adding a media level barrier 660 which sets the media level to a desired target. To achieve the desired effect, the media level is set higher than the viewing glass 650, but below the bottom surface of the cover, thus forming the desired air cavity 670. In an embodiment the media level barrier 660 is placed at the media outlet 630.

FIG. 6E shows an example perfusion observation chamber 600E according to an embodiment. Via the media inlet 610 biological media flows above the well array chip 640, and fills the media chamber up to a level permitted by the media level barrier 660, which is above the depth to which the viewing glass 650 is submerged. This allows for excess media to flow out of the media chamber thereby ensuring a constant supply of fresh media over the well array chip 640 thereby maintaining the viability of trapped cells within the well array chip. FIG. 6F provides an enlarged view of the media level barrier 660 and the air cavity 670 in an embodiment.

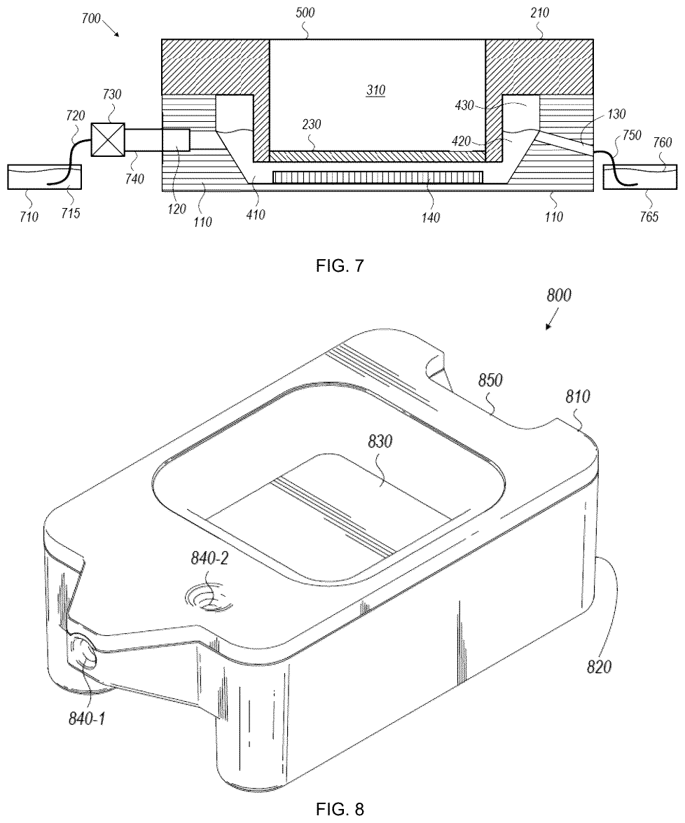

FIG. 7 is an example schematic block diagram of a system 700 employing a perfusion observation chamber 500 according to another embodiment. A fresh biological media 715 (in short, fresh media) may be provided from a source container 710. A pump 730 is connected by an input pipe 740 to the inlet 120 of the perfusion observation chamber 500. The pump is further connected to a feeding pipe 720 that is immersed in the fresh media 715. A draining container 760 may be used for the collection of the used biological media 765 (in short, used media). To the outlet of the perfusion observation chamber 500, there is connected a draining pipe 750 that is extended to dispose of the used media 765 by directing it from the outlet 130 via the draining pipe 750 into the draining container 760. It should be understood that both the source container 710 and the draining container 760 shown herein are for illustration purposes only and many other forms of sources and draining options may be used without departing from the scope of the disclosed embodiments.

When in operation, the pump 730 is operated to draw fresh media 715 from the source container 710, and into the perfusion observation chamber 500. The chamber will be filled with media in the media chamber 410 and restricted, as explained in greater detail herein, to ensure that an air bubble escape chamber remains open to allow for air bubbles to escape therein. Due to the continuous operation of the pump 730, fresh media continues to enter the media chamber 410 ensuring that the channel formed between the viewing glass 230 and the top surface of the well array chip 140 is continuously filled with fresh media, as by design, and as explained herein, the media barrier is at a height that is above the bottom surface of the viewing glass 230. This ensures the necessary supply cells trapped in wells of the well array chip 140, while providing the quality imaging capability from the observation chamber 310.

FIG. 8 is an example of a three-dimensional illustration of a perfusion observation chamber 800 for a well array chip according to an embodiment. The perfusion observation chamber 800 includes a top part 810 and a bottom part 820 where the top part 810 is mounted on top of the bottom part 820. The top part 810 further includes a chamber at the bottom of which there is a viewing glass 830. A first inlet 840-1 is adapted to allow for a flow of media into the internal chamber (not seen in this illustration) that is created when the top part 810 is mounted on top of the bottom part 820. In an embodiment, a second inlet 840-2 is provided, wherein the second inlet 840-2 is part of the top part 810. Media may flow from the second inlet 840-2 into the internal chamber. In an embodiment, the media flowing from the first inlet 840-1 into the internal chamber is different from the media flowing into the internal chamber from the second inlet 840-2. An outlet area 850 (not seen in this view) allows the media that enters from either of the first inlet 840-1 or the second inlet 840-2 to exit the perfusion observation chamber 800.

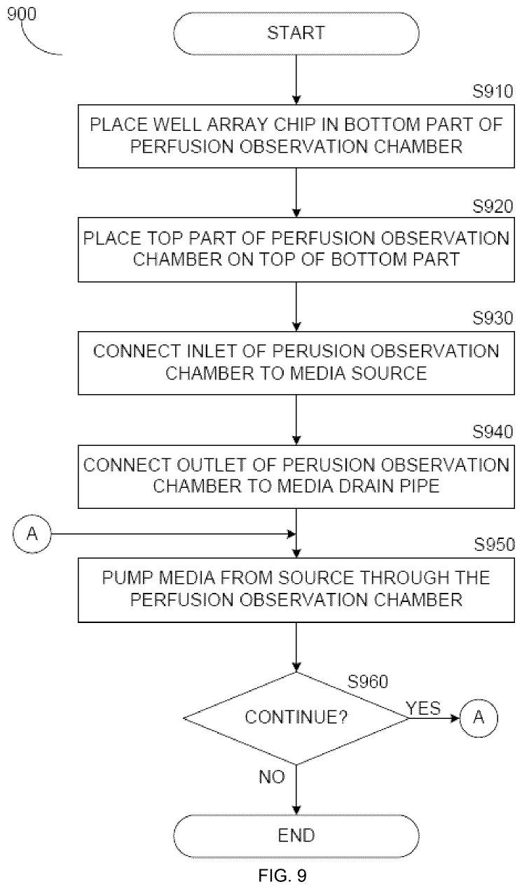

FIG. 9 is an example flowchart 900 for an operation of the system employing a perfusion observation chamber 500 according to another embodiment. For simplicity, reference numbers that were introduced in FIGS. 1, 5, and 7 are used, but do not limit the scope of the disclosed embodiments. At S910, a well array chip, for example well array chip 140, is placed within the bottom part 100 of the perfusion observation chamber 500.

At S920, the top part 200 of the perfusion observation chamber 500 is placed on top of the bottom part 100.

At S930, the inlet 120 of the perfusion observation chamber 500 is connected to a biological media source, for example, via a pipe 740 to a pump 730, then via a feeding pipe 720 to a fresh media 715.

At S940, the outlet 130 is connected to a draining pipe 750. This is done for the purpose of discarding used media 765 appropriately.

At S950, a pump, for example, pump 730 begins to pump fresh media through the perfusion observation chamber 500, as explained in detail herein.

At S960, it is checked whether the process of providing fresh media into the chamber 420 should continue, and if so, execution continues with S950; otherwise, execution terminates. One of ordinary skill in the art would appreciate that, in an embodiment, if there is no need to provide fresh media into the chamber 420, the addition of media may cease for at least a period of time.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; A and B in combination; B and C in combination; A and C in combination; or A, B, and C in combination.

All examples and conditional language recited herein are intended for pedagogical purposes to aid the reader in understanding the principles of the disclosed embodiment and the concepts contributed by the inventor to further the art and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

What is claimed is:

1. An observation chamber comprising:
a bottom part configured to accept a well array chip, each well therein designed to accept at least a biological cell;
one or more sidewalls extending from the bottom part;
a top part comprising a viewing glass;
wherein the bottom part, the one or more sidewalls, and the top part enclose therein a first media chamber; and
wherein the first media chamber is configured to receive a biological media such that the viewing glass is submerged therein.

2. The observation chamber of claim 1, further comprising:
one or more inlets for filling the first media chamber with the biological media.

3. The observation chamber of claim 1, further comprises:
a fastening mechanism configured to allow placement of the well array chip within the chamber and to close the first media chamber thereafter.

4. The observation chamber of claim 3, wherein the fastening mechanism is a mechanical mechanism.

5. The observation chamber of claim 3, wherein the fastening mechanism uses at least in part magnetic forces.

6. The observation chamber of claim 3 wherein the fastening mechanism comprises:
a plurality of screws that secure the first media chamber.

7. The observation chamber of claim 3, wherein the fastening mechanism further comprising:
a seal.

8. The observation chamber of claim 1, further comprising:
one or more outlets for removal of the biological media from the first media chamber.

9. The observation chamber of claim 1, wherein the first media chamber has a gap between the viewing glass and the well array chip to allow for flow of the biological media, wherein the gap is a predetermined distance.

10. The observation chamber of claim 9, wherein the predetermined distance provides a uniform layer of the biological media.

11. The observation chamber of claim 1, further comprising:
an air escape chamber, wherein the air escape chamber is configured to provide an escape path for air bubbles trapped within the biological media such that image quality is maintained.

12. The observation chamber of claim 1, further comprising:
at least a securing element for attachment of the well array chip to the bottom part.

13. The observation chamber of claim 1, further comprising:
a level barrier configured to limit a level of the biological media within the first media chamber.

14. The observation chamber of claim 13, wherein the level barrier is designed to ensure that the viewing glass is submerged in the biological media.

15. The observation chamber of claim 13, wherein the level barrier is designed to ensure an escape path for air bubbles trapped in the biological media.

16. The observation chamber of claim 1, wherein at least a portion of the first media chamber is coated with a hydrophobic material.

17. The observation chamber of claim 1, wherein the viewing glass is made of an optically transparent material.

18. The observation chamber of claim 1, wherein the viewing glass is coated by at least one of: an anti-reflective layer on at least one side of the viewing glass and an optical filter layer on the at least one side of the viewing glass.

19. The observation chamber of claim 1, further comprises:
a first pump connected to an inlet of one or more inlets of the observation chamber, wherein the first pump is configured to provide the biological media to the inlet of the observation chamber; and
a draining pipe connected to an outlet of one or more outlets of the observation chamber.

20. The observation chamber of claim 19, further comprises:
a second pump, wherein the second pump is connected to the draining pipe.
